(19) United States
(12) Patent Application Publication
(10) Pub. No.: US 2007/0059392 A1
(43) Pub. Date: Mar. 15, 2007

(54) COX inhibitor

(76) Inventor: Peter Rohdewald, Munster (DE)

Correspondence Address: Mintz, Levin, Cohn, Ferris,;Glovsky and Popeo, P.C., The Chrysler Center, 666 Third Avenue, 24th Floor, New York, NY, 10017, US

(21) Appl. No.: 11/300,119

(22) Filed: Dec. 13, 2005

Related U.S. Application Data
(60) Provisional application No. 60/718,261, filed on Sep. 15, 2005.

(51) Int. Cl. A61K 36/13; A61K 31/7048; A61K 31/353

(52) U.S. Cl. 424/770; 514/27; 514/456

(57) ABSTRACT

Methods and compositions for inhibition of both COX-1 and COX-2 are provided by this disclosure. The methods and compositions on the invention are effective for providing relief from pain.

### RELATED APPLICATIONS

This application claims the benefit of priority to U.S. application 60/718,261 filed Sep. 15, 2005. All patents, patent applications and references cited in this disclosure are hereby incorporated by reference in their entirety.

### BACKGROUND

Treatment of Pain

There are at least five main categories of drugs available to treat pain. These include analgesics (e.g. acetaminophen); salicylates (e.g. aspirin) and other non-steroidal anti-inflammatory drugs (NSAID, e.g., ibuprofen and naproxen); opoid drugs (e.g. codeine and morphine); corticosteroids; and adjuvant agents (e.g. antidepressants, anticonvulsants).

NSAIDs provides pain relieve by inhibiting the activities of the enzyme cyclo-oxygenase (COX). COX acts on arachidonic acid to generate prostaglandin G₂ (PGG₂), and prostaglandin (PGH₂). PGG₂ and PGH₂ are not stable and are converted into prostacyclin (PGI₂) by PGI₂ synthase, thromboxane (TXA₂) by TXA₂ synthase, and stable prostaglandins PGD₂, PGE₂, and PGF₂′. Also, the enzyme lipoxygenase can act on arachidonic acid to generate leukotrienes from 5-HETE. The end result of this pathway is inflammation and pain. It follows that inhibition of COX will also provide pain suppression.

The COX enzyme exists in two isoforms, COX-1 and COX-2. COX-1 is constitutively expressed and involved in the maintenance of prostaglandin mediated physiological functions. In contrast, COX-2 is inducible and is present in negligible amounts under normal conditions but is substantially induced in vivo under inflammatory condition. The most widely available NSAIDs are non-selective COX inhibitors which inhibits COX-1 and COX-2.

Although NSAID are widely accepted as effective agents for controlling pain, their administration can lead to the development of undesirable side effects such as stomach pain or heartburn, stomach ulcers, bleeding, headaches and dizziness, ringing in the ears, allergic reactions, and liver or kidney problems. Attempts to develop NSAIDs with fewer side effects have met with only limited success. For example, the recently developed cyclooxygenase-2 (COX-2) inhibitors show a reduced tendency to produce gastrointestinal ulcers and erosions, but it is associated with significant cardiovascular problems and other side effects.

For these reasons, patients with inflammatory disease or pain have increasingly began to seek natural NSAIDs which can provide some or all of the benefits of NSAID but with reduced or eliminated side effect.

Proanthocyanidins and Anthocyanidins

Proanthocyanidins, especially proanthocyanidins in the form of proanthocyanidins rich extracts such as Pycnogenol®, have been described in numerous references including, for example, Passwater, R. A. The New Superantioxidant Plus, Keats Publishing Inc., New Canaan, Conn. USA, 1992, Passwater, R. A., All About Pycnogenol, Avery Publishing Group, Garden City Park, N.Y., 1998, Passwater, R. A. and Kandaswami, C., Pycnogenol The Super Protector Nutrient, Keats Publishing Inc., New Canaan, Conn. USA, 1994, Passwater, R. A. Pycnogenol for Superior Health, McCleery and Sons Publishing, Fargo, N. Dak. USA, 2001, and Passwater, R. A. Pycnogenol for Superior Health, Editions Stylum, Switzerland, 2001.

Proanthocyanidins got their name from the fact that these molecules, which are colorless to brownish substances, would turn into deeply colored anthocyanidins after oxidative hydrolysis in the laboratory. Since proanthocyanidins turn into anthocyanidins, they are considered as pro-forms of anthocyanidins (i.e., the colorless precursors of anthocyanidins).

While proanthocyanidins and anthocyanidins are related chemically, there are striking differences. Anthocyanidins are the colored substances contained in blue or red flowers and fruits, whereas proanthocyanidins (light brownish substances) are contained in seeds or skins of fruits or in barks. Anthocyanidins are small molecules, whereas proanthocyanidins represent biopolymers, consisting of several units as for example of catechin and epicatechin in procyanidins, a subgroup of the proanthocyanidins. Most importantly, proanthocyanidins and anthocyanidins do not interconvert in any significant manner inside a mammalian body.

While proanthocyanidins are precursors of anthocyanidins, the consumption of proanthocyanidins does not lead to delivery of anthocyanidins to a mammal—even after the proanthocyanidins are metabolized in the body. After consumption, proanthocyanidins may be converted into its subunits such as catechin and epicatechin and its metabolites, either valerolactones of phenolic acids (Grosse Düweler K, Rohdewald P. Urinary metabolites of French maritime pine bark extract in humans. Pharmazie, 2000. 55: p. 364-368). Importantly, no significant amounts of anthocyanidins are produced in the body after proanthocyanidins consumption (Id.).

The reason that no significant amount of anthocyanidins are produced in response to proanthocyanidins consumption lies in the fact that the metabolization process does not involve an oxidative acid hydrolysis—a process which is produced in the laboratory but which contains conditions not compatible to the well being of mammals. Consistent with this observation, no formation of cyanidium ions was observed in gastric juice (Rios L Y et al., Cocoa procyanidins are stable during gastric transit in humans. Am J Clin Nutr, 2002. 76: p. 1106-1110).

U.S. Pat. No. 6,818,234 B1 teaches the inhibition of cyclooxygenase by fruit extracts containing anthocyanidins. From the findings listed in U.S. Pat. No. 6,818,234 B1 it cannot be deduced that proanthocyanidins inhibit cyclooxygenases in vivo, because, as discussed above, the metabolization of proanthocyanidins in human body does not produce significant amounts of anthocyanidins. Instead, metabolization of procyanidins produces substances which lacks the flavylium structure which is characteristic for anthocyanidins.

### BRIEF DESCRIPTION OF THE INVENTION

There is evidence from several studies that supplementation with proanthocyanidins rich French maritime pine bark extract (Pycnogenol®) improves inflammatory symptoms in vivo. However, the molecular pharmacological basis for the observed effects has not been fully uncovered. Direct inhibitory effects of plant extracts or components upon cyclooxygenase (COX) activity have been repeatedly reported, but the question remained whether sufficiently high in vivo concentrations of bioactive compounds could be achieved in humans.

To determine if a proanthocyanidins rich extract can inhibit the activities of COX-1 and/or COX-2, we performed a study of plasma samples taken from of human volunteers after intake of French maritime pine bark extract. This study is based on the principle that the plasma sample of a subject which ingested a proanthocyanidins rich pine bark extract would contain any bioavailable active principles from the extract. To test this theory, we obtained blood samples before and after five days administration of 200 mg Pycnogenol to five healthy humans. The plasma moderately inhibited both COX-1 and COX-2 activity ex vivo. In a second approach, ten volunteers received a single dose of 300 mg Pycnogenol. Only 30 min after ingestion of the pine bark extract the plasma samples induced a statistically significant increase in the inhibition of both COX-1 (p<0.02) and COX-2 (p<0.002). This suggests a strikingly rapid bioavailability of bioeffective compounds after oral intake of the extract. Thus, we provide evidence that Pycnogenol exerts effects by inhibition of eicosanoid generating enzymes which is consistent with reported clinical anti-inflammatory and platelet inhibitory effects in vivo.

One embodiment of the invention is directed to a method of inhibiting both COX-1 and COX-2 activity in a mammal. COX-1 and COX-2 activity may be measured in the blood (i.e., plasma) of the mammal according to known procedures (See, Example Section). The method involves the step of administering to the mammal a composition comprising proanthocyanidins. The composition may be, for example, a proanthocyanidins rich plant extract such as Pycnogenol. While all mammals may be treated with the methods of the invention, the preferred mammals for treatment are humans and valuable livestocks and pets such as cows, pigs, horses, goats, dogs and cats and the like.

By applying the methods of the invention, both COX-1 and COX-2 activity in a mammal may be reduced simultaneously. In particular, COX-1 and COX-2 activity may be reduced in the blood of a mammal. The amount of COX-1 and COX-2 activity reduction may be at least 10%. That is, a mammal treated with the methods of the invention will have a reduction of COX-1 activity of at least 10% and a reduction of COX-2 activity of at least 10%. In a preferred embodiment, a mammal treated with the methods of the invention will have a reduction of COX-1 activity of at least 20% and a reduction of COX-2 activity of at least 20%. In another embodiment, a mammal treated with the methods of the invention will have an increase of COX-1 inhibitory activity of at least 10% and an increase in COX-2 inhibitory activity of at least 10%. In a preferred embodiment, the treated mammal will have an increase of COX-1 inhibitory activity of at least 20% and an increase of COX-2 inhibitory activity of at least 20%. The COX activity and COX inhibitory activity may be measured, for example, in the blood or plasma of a mammal.

In another preferred embodiment, the composition administered to a patient comprises an analgesic dose of proanthocyanidins which can reduce pain in the mammal. The proanthocyanidins may be in the form of a proanthocyanidins rich extract (e.g., Pycnogenol). The pain may be any pain in the mammal including, for example, headache, muscle pain, joint pain and the like.

The composition of the invention may be administered to a mammal using any method including enteral (e.g., oral) administration, topical administration and parenteral (e.g., injection) administration. Naturally, the composition of the invention may be a composition for the various administration methods mentioned. When the composition is administered orally, it may be in the form of pills, drinks, powders, food additives, powders, capsules, time-release-capsules and the like.

The daily dosage of proanthocyanidins per mammal may be between 10 mg to 10 grams, between 20 mg to 5 grams, or between 50 mg to 1 gram. Administration may be for a period of at least 7 days, at least 14 days, at least 21 days or as long as COX-1 and COX-2 inhibition is necessary (e.g., daily maintenance dose). COX-1 and COX-2 inhibition may be necessary until, for example, the symptom of pain is gone.

The proanthocyanidins referred to in this disclosure may be from a proanthocyanidins-rich plant extract comprising 10% to 100% proanthocyanidins. The extract may be an extract from a plant material such as, for example, an extract from pine bark. The pine bark may be from a pine of the Pinus pinaster species.

### BRIEF DESCRIPTION OF THE FIGURES

FIG. 1 depicts an increase in inhibition of cyclooxygenase (COX) 1 and COX-2 enzymatic activity in the plasma of five healthy volunteers after five days intake of 200 mg French maritime pine bark extract (Pycnogenol) per day. The increase of inhibitory activity towards COX-1 as observed in three volunteers, inhibition of COX-2 activity was obvious in two volunteers (not statistically significant, Wilcoxon matched pairs signed rank test).

FIG. 2: depicts an increase in inhibition of cyclooxygenase (COX) 1 and COX-2 enzymatic activity by plasma of ten healthy volunteers 30 minutes after intake of 300 mg French maritime pine bark extract (Pycnogenol). The increase of inhibitory activity towards COX-1 and COX-2 was statistically significant (p<0.02 and p<0.002, respectively; Wilcoxon matched pairs signed rank test).

### DETAILED DESCRIPTION OF THE INVENTION

It is understood that the term “pine bark extract” in this disclosure refers to a French maritime pine bark extract which is, for example, commercially available as Pycnogenol® (Horphag). The terms “Pycnogenol®”, “pine bark extract” and “French maritime pine bark extract” are interchangeable in this disclosure.

Pinus pinaster (P. pinaster) and Pinus maritima (P. maritime), are understood to refer to the same organism. Hence, these terms are interchangeable.

Proanthocyanidins designates a group of flavonoids that includes the subgroups procyanidins, prodelphinidins and propelargonidins. Proanthocyanidins are homogeneous or heterogeneous polymers consisting of the monomer units catechin or epicatechin, which are connected either by 4-8 or 4-6 linkages, to the effect that a great number of isomer proanthocyanidins exist. Typically, the proanthocyanidins oligomers have a chain length of 2-12 monomer units. Proanthocyanidins may be synthesized or extracted from a plant material. Nonlimiting examples of plant material sources of proanthocyanidins include grape seeds, grape skin, pine barks, ginkgo leaves, peanuts, and cocoa beans, tamarind, tomato, peanut, almond, apple, cranberry, blueberry, tea leaves.

A well-known product containing proanthocyanidins, which is available in trade as a preparation of a food supplement under the name Pycnogenol®, is an extract of the maritime pine bark (Pinus pinaster). Pycnogenol®, the extract from French maritime pine bark (Pinus pinaster) is a registered trademark belonging to Horphag Research, Ltd. Pycnogenol® is a standardized bark extract of the French maritime pine Pinus pinaster, Aiton, subspecies Atlantica des Villar (Pycnogenol®, Horphag Research Ltd., UK). The quality of this extract is specified in the United States Pharmacopeia (USP 28) [1]. Between 65-75% of Pycnogenol® are procyanidins comprising of catechin and epicatechin subunits with varying chain lengths [2]. Other constituents are polyphenolic monomers, phenolic or cinnamic acids and their glycosides [2].

The effects of Pycnogenol have been explored in multiple in vitro and various animal and human in vivo studies [2, 3]. A considerable antioxidant activity was detected in simple in vitro or cell culture assays. Additionally to its radical scavenging activity an inhibition of NF-κB-dependent gene expression and decrease of the activity of various enzymes was observed for the Pycnogenol extract [3]. In vivo studies revealed distinct pharmacological actions [2]. These effects include anti-inflammatory and cardiovascular bioefficacy. Thrombus formation was prevented in passengers of long-haul flights after intake of Pycnogenol® [4]. Supplementation of cardiovascular patients with maritime pine bark extract reduced platelet aggregation and improved microcirculation [5]. Anti-inflammatory effects of maritime pine bark extract were observed in experimental inflammatory bowel disease in animals [6]. Reduced urine [7] or serum [8] leukotriene concentrations were measured after Pycnogenol® administration while asthma symptom scores and pulmonary function improved in asthma patients [7, 8]. Analgesic effects of Pycnogenol® were observed in women with menstrual pain [9].

Thus, there is evidence from several in vivo studies that supplementation with French maritime pine bark extract improves inflammatory symptoms. It is tempting to explain the cellular or molecular mechanism of the observed in vivo effects with the results of effect studies on biological systems in vitro. There are two issues that advise caution in interpretation and conclusions from in vitro effects. One issue is to consider whether the concentrations of the plant extract applied to cell cultures or enzymes in vitro would be realistic or achievable in vivo concentrations. In most cases this has to be denied due to the fact that plant extracts are typically not completely bioavailable and only certain fractions or components can be expected to be absorbed. French maritime pine bark extract, for example, comprises of high molecular weight components that have not been shown to pass biological membranes in the gastrointestinal tract in quantity. The other issue that needs a second thought is that bioeffective compounds do not necessarily need to be present in the original extract, but might be formed in vivo due to intestinal bacterial and/or hepatic metabolism. After ingestion of Pycnogenol®, for example, two metabolites derived from catechin were detected in human urine, δ-(3,4-dihydroxy-phenyl)-γ-valerolactone and δ-(3-methoxy-4-hydroxy-phenyl)-γ-valerolactone [10]. We recently demonstrated that these metabolites inherit an antioxidant activity as well as the potential to inhibit release and enzymatic activity of matrix metalloproteinase 9 in vitro [11]. Yet, it remained elusive whether sufficiently high in vivo concentrations of these metabolites could be attained.

A methodological approach that would consider both the absorption and possible metabolism of plant extract components would involve laboratory animals or human volunteers who donate blood samples before and after ingestion of the plant extract. The plasma samples must contain any bioavailable active principles of the extract. These plasma samples could be used in all kind of assays to uncover molecular pharmacological effects ex vivo in a realistic setting. The purpose of the present study was to determine a possible direct inhibition of the enzymatic activity of cyclooxygenase (COX) 1 and COX-2 ex vivo by plasma samples after intake of regular doses of French maritime pine bark extract by human volunteers. We aimed to contribute to the explanation of the platelet aggregation inhibition, anti-inflammatory and analgesic effects of Pycnogenol® observed in vivo.

Experimental methods and the results of our experiments are discussed in the Example section.

Discussion

The in vivo effects of plant constituents and extracts are studied with increasing interest [13, 14], but the elucidation of the molecular basis of biological or clinical effects remains a challenge. Bioavailability and metabolism factors must be considered for valid explanations of bioefficacy. Further complicating factors arise from the knowledge that plant extracts comprise of a complex mixture of various components and often enough it is not clear whether a single compound or a mixture of related compounds is responsible for the effects. Sometimes lead effect compounds might be identified and defined active principles might be analyzed for precise molecular pharmacological effects in vitro.

In the present study we employed an experimental approach that considered absorption and metabolism of complex constituents of French maritime pine bark extract to shed some light into the molecular basis of documented clinical anti-inflammatory and platelet aggregation inhibitory activity [2]. We here provide the first report on active principles in human plasma after intake of Pycnogenol that statistically significantly inhibited the enzymatic activity of COX-1 and COX-2.

Regular doses of 200 mg maritime pine bark extract were administered to five human volunteers over a period of five days. We elucidated the increase of COX-1 and COX-2 inhibition by the participants' plasma samples. Though we observed inhibitory activity the mean increase of cyclooxygenase inhibition failed to be statistically significant due to the pronounced variations between individuals and the limited number of participating volunteers. Significantly higher PGF₂ₐ, concentrations were measured in the presence of COX-2 indicating that the enzymatic activity of COX-2 was less influenced by Pycnogenol® metabolites or constituents.

In a second approach 300 mg Pycnogenol was administered as a single dose to ten study participants. We sought to determine how fast an effect would be measurable in vivo. Only 30 min after ingestion of the pine bark extract the plasma samples induced a statistically significant increase in the inhibitory activity of both COX-1 (p<0.02) and COX-2 (p<0.002). This suggests a strikingly rapid bioavailability of bioeffective compounds after oral intake of the extract.

Direct inhibitory effect of plant extracts or components upon cyclooxygenase activity have been repeatedly reported (e.g. [15-19]). After Pycnogenol intake the inhibition induced by volunteers' plasma samples towards COX-2/COX-1 indicated a ratio of 8±3 after repeated ingestion and 10±6 after single dose administration. Though these ratios were not calculated from IC₅₀ concentrations as usually reported in literature they are suggestive of a nonselective inhibition of COX-1 and COX-2 [12]. This is consistent with reports of various flavonoids characterized as mixed COX-1/COX-2 inhibitors [15]. COX-2 specific inhibition with a COX-2/COX-1 ratio below 1 was reported for resveratrol and analogues [16, 20] or butterbur extracts [19]. Though COX-2 plays a pivotal role in inflammatory processes COX-1/COX-2 nonselective inhibitors appear more favorable in some respects [15, 21].

Since those investigations of a potential inhibition of COX-1/COX-2 either employed plant extracts or single isolated flavons or flavanols the question remained whether sufficiently high in vivo concentrations of these flavonoids could be achieved in humans. We now provide the novel information that after intake of French maritime pine bark extract plasma concentrations of active principles were ex vivo effective in inhibition of eicosanoid generating enzymes. These results contribute to explain clinical pharmacological effects of Pycnogenol such as reduction of platelet aggregation [5], anti-inflammatory effects in asthma patients [7, 8], pain relief [9] and reduction of UV-induced erythema formation [22].

To summarize, moderate molecular pharmacological effects were observed ex vivo with plasma of volunteers after Pycnogenol administration and these effects are consistent with reported clinical anti-inflammatory and platelet inhibitory effects in vivo. The next challenge is to identify the responsible active principle(s) that are rapidly bioavailable in human plasma samples.

### EXAMPLE

### Example 1

### Determining the COX-L and COX-2 Inhibitory Effects of Pycnogenol

Patients

Five and ten, respectively, healthy volunteers aged 18 to 30 years participated in this study. After 24 hours of a diet free of flavonoids (no vegetables, fruits and fruit juices or marmalades, tea, coffee, cocoa, wine and beer) blood samples were drawn to obtain basal values. Subsequently, five of the volunteers took tablets containing 200 mg standardized maritime pine bark extract (Pycnogenol®, Horphag Research Ltd., UK) every morning for five days to reach steady state conditions of constituents and/or metabolites of Pycnogenol. Four hours after the last intake of Pycnogenol on day five a second blood sample was obtained from each volunteer. Again, a 24 hour period of a diet free of flavonoids preceded this blood sampling. Additionally, ten volunteers took tablets containing 300 mg standardized maritime pine bark extract as a single dose. Before and 30 min after intake of Pycnogenol blood samples were obtained from each volunteer. All blood samples were centrifuged and plasma was aliquoted, shock frozen and stored at −80° C. until further analysis.

Determination of COX-1 and COX-2 Activity by ELISA

The COX-1 (ovine) and COX-2 (human recombinant) inhibitory assay was carried out using a COX Inhibitor Screening Assay Kit (Cayman Chemicals, Ann Arbor, Mich., USA), according to the manufacturer's protocol. Briefly, heme and COX enzymes, COX-1 and COX-2, respectively, were added to test tubes containing COX reaction buffer (0.1 M Tris-HCl, pH 8.0, containing 5 mM EDTA and 2 mM phenol). The mixture was vortexed and exposed to either reaction buffer or plasma sample for 10 min at 37° C. Acetylsalicylic acid (aspirin), the irreversible COX-1 inhibitor, was used at a concentration corresponding to its reported IC₅₀ concentration (1.67 μM for COX-1 and 278 μM for COX-2 according to [12]) as a positive control. Subsequently, arachidonic acid solution was added to start the cyclooxygenase reaction. After incubation for 2 min and 37° C. 1 M hydrochloric acid was added to terminate the enzyme catalytic reaction followed by chemical reduction of prostaglandin (PG) PGHZ₂ to PGF_{2α}, with saturated stannous chloride solution for 5 min at room temperature. The reaction products were stable for one week at 0-4° C. The COX activity was measured based on the amount of PGF_{2α}, generated in the reaction tube and detected by the enzyme immunoassay kit using a standard curve. The inhibitory activity of individual plasma samples before Pycnogenol intake was compared to the COX inhibition induced by plasma samples after Pycnogenol supplementation. The difference was expressed as increase in COX inhibitory activity.

Statistical Analysis

Statistical analysis (Wilcoxon matched pairs signed rank test) was performed using the GraphPad prism software (GraphPad Software Inc., San Diego Calif., USA). Significance was defined as p<0.05.

Results

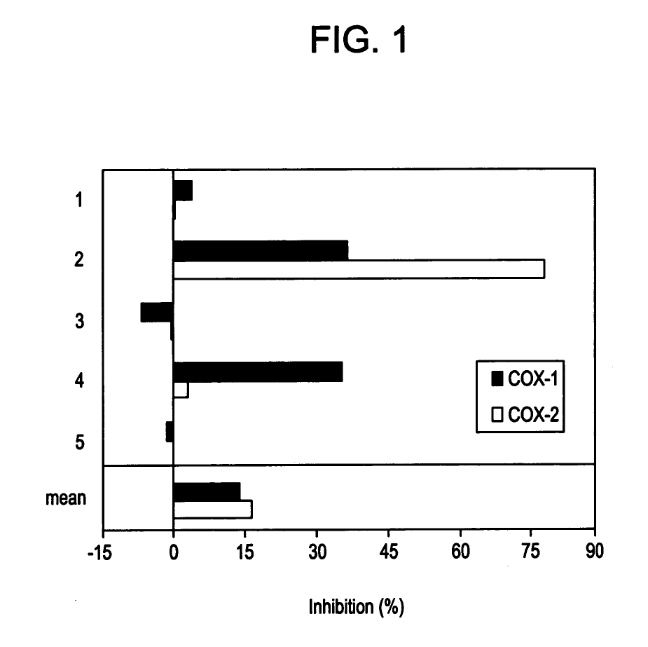

After repeated intake of a daily dose of 200 mg French maritime pine bark extract incubation of five volunteers' plasma samples with COX-1 and COX-2, respectively, revealed direct inhibitory effect on cyclooxygenase activity (FIG. 1). An inhibition of COX-1 was obvious in three of five participants. The mean increase of cyclooxygenase inhibition after administration of Pycnogenol to volunteers was 13.8±18.1% for COX-1 and 16.5±35.3% for COX-2. The increase of cyclooxygenase inhibition was not statistically significant (Wilcoxon matched pairs signed rank test). The mean PGF_{2α}, concentrations determined after incubation with plasma samples obtained before Pycnogenol ingestion were 315±122 ng/mL, after intake of Pycnogenol the concentrations were modestly reduced to 243±76 ng/mL. The interindividual variability was pronounced. Inhibition experiments with COX-2 generally revealed higher PGF_{2α}, concentrations indicating that the enzymatic activity of COX-2 was less influenced by Pycnogenol metabolites or constituents. An inhibition of COX-2 was obvious only in two of five participants and interindividual differences were marked. The mean PGF_{2α} concentrations determined after incubation with plasma samples obtained before Pycnogenol ingestion were 3761±4750 ng/mL, after intake of Pycnogenol the concentrations were reduced to 2021±1378 ng/mL. The COX-2/COX-1 ratio of inhibitory activity of plasma samples was determined as the ratio of prostaglandin concentrations after intake of maritime pine bark extract in individual volunteers. The COX-2/COX-1 ratio was above 1 for all participants, the mean ratio was 8±3 suggesting a nonselective inhibition of both COX-1 and COX-2.

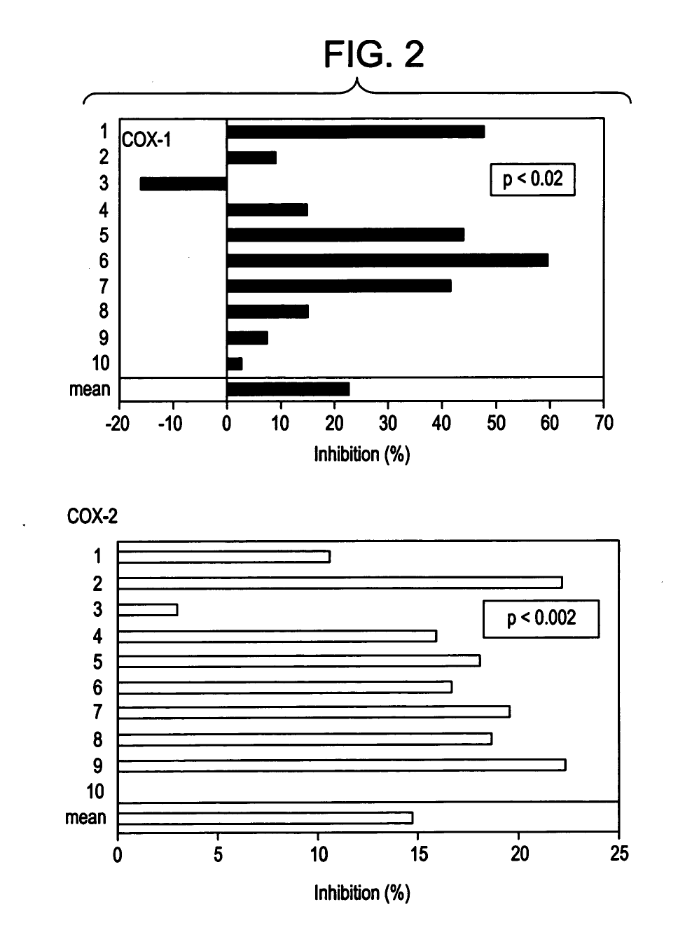

The evaluation of COX-1 and COX-2 inhibition after ingestion of French maritime pine bark extract implied that biologically active components or metabolites were present in the plasma of some volunteers. This needed to be verified and thus a higher number of volunteers were recruited. Ten participants took a single dose of 300 mg Pycnogenol. Plasma samples of one volunteer were screened for earliest inhibitory activity towards COX-1 and COX-2. Surprisingly, inhibition of enzymatic activity was already measurable after 30 min. The plasma samples obtained after 30 min from all volunteers were incubated with COX-1 and COX-2, respectively. As observed after repeated intake of maritime pine bark extract a direct inhibitory effect on cyclooxygenase activity was detected again (FIG. 2). An increased ex vivo inhibition of COX-1 after ingestion of the extract was obvious in nine of ten participants, an increased inhibition of COX-2 was produced by the plasma of all volunteers. The mean increase of cyclooxygenase inhibition after administration of Pycnogenol to volunteers was 22.5±24.1% for COX-1 and 14.7±7.8% for COX-2. These inhibitory activities were statistically significant (p<0.02 for COX-1 and p<0.002 for COX-2; Wilcoxon matched pairs signed rank test). The mean PGF_{2α}, concentrations determined after incubation with plasma samples obtained before Pycnogenol ingestion were 305±135 ng/mL, after intake of Pycnogenol the concentrations were modestly reduced to 253±122 ng/mL. The interindividual variability was pronounced. Inhibition experiments with COX-2 again revealed higher PGF_{2α}. The mean PGF_{2α} concentrations determined after incubation with plasma samples obtained before Pycnogenol ingestion were 1906±590 ng/mL, after intake of Pycnogenol the concentrations were 2034±196 ng/mL. The COX-2/COX-1 ratio was above 1 for all participants, the mean ratio was 10±6 confirming a nonselective inhibition of both COX-1 and COX-2.

### REFERENCES

[1] Maritime Pine Extract. In: United States Pharmacopeia. Rockville: United States Pharmacopeial Convention, Inc.; 2005. pp. 21 15-2116.
[2] Rohdewald P. A review of the French maritime pine bark extract (Pycnogenol), a herbal medication with a diverse clinical pharmacology. Int J Clin Pharmacol Ther 2002; 40: 158-168.
[3] Packer L, Rimbach G, Virgili F. Antioxidant activity and biologic properties of a procyanidin-rich extract from pine (Pinus maritima) bark, pycnogenol. Free Radic Biol Med 1999; 27:704-724.
[4] Belcaro G, Cesarone M R, Rohdewald P, Ricci A, lppolito E, Dugall M, Griffin M, Ruffini I, Acerbi G, Vinciguerra M G, Bavera P, Di Renzo A, Errichi B M, Cerritelli F. Prevention of venous thrombosis and thrombophlebitis in long-haul flights with pycnogenol. Clin Appl Thromb Hemost 2004; 10:373-377.
[5] Wang S, Tang D, Zhao Y, Gao G, Gao X, Hu L. The effects of Pycnogenol on the mircocirculation, platelet function and ischemic myocardium in patients with coronary artery diseases. Eur Bull Drug Res 1999; 7:19-25.
[6] Mochizuki M, Hasegawa N. Therapeutic efficacy of pycnogenol in experimental inflammatory bowel diseases. Phytother Res 2004; 18:1027-1028.
[7] Lau B H, Riesen S K, Truong K P, Lau E W, Rohdewald P, Barreta R A. Pycnogenol as an adjunct in the management of childhood asthma. J Asthma 2004; 41:825-832.
[8] Hosseini S, Pishnamazi S, Sadnadeh S M, Farid F, Farid R, Watson R R. Pycnogenol® in the Management of Asthma. J Med Food 2001; 4:201-209.
[9] Kohama T, Suzuki N. Ohno S, lnoue M. Analgesic efficacy of French maritime pine bark extract in dysmenorrhea: an open clinical trial. J Reprod Med 2004; 49:828-832.
[10] Diiweler K G, Rohdewald P. Urinary metabolites of French maritime pine bark extract in humans. Pharmazie 2000; 55:364-368.
[11] Grimm T, Schsfer A, Hogger P. Antioxidant activity and inhibition of matrix metalloproteinases by metabolites of maritime pine bark extract (pycnogenol). Free Radic Biol Med 2004; 36:811-822.
[12] Mitchell J A, Akarasereenont P. Thiemermann C, Flower R J, Vane J R. Selectivity of nonsteroidal antiinflammatory drugs as inhibitors of constitutive and inducible cyclooxygenase. Proc Natl Acad Sci USA 1993; 90:11693-11697.
[13] Manach C, Williamson G, Morand C, Scalbert A, Remesy C. Bioavailability and bioefficacy of polyphenols in humans. I. Review of 97 bioavailability studies. Am J Clin Nutr 2005; 81:230S-242S.
[14] Williamson G, Manach C. Bioavailability and bioefficacy of polyphenols in humans. II. Review of 93 intervention studies. Am J Clin Nutr 2005; 81:243S-255S.
[15] Kim H P, Son K H, Chang H W, Kang S S. Anti-inflammatory plant flavonoids and cellular action mechanisms. J Pharmacol Sci 2004; 96:229-245.
[16] Seaver B, Smith J R. Inhibition of COX isoforms by nutraceuticals. J Herb Pharmacother 2004; 4:11-18.
[17] Selvam C, Jachak S M. A cyclooxygenase (COX) inhibitory biflavonoid from the seeds of Semecarpus anacardium. J Ethnopharmacol 2004; 95:209-212.
[18] Cavin C, Delannoy M, Malnoe A, Debefve E, Touche A, Courtois D, Schilter B. Inhibition of the expression and activity of cyclooxygenase-2 by chicory extract. Biochem Biophys Res Commun 2005; 327:742-749.
[19] Fiebich B L, Grozdeva M, Hess S, Hull M, Danesch U, Bodensieck A, Bauer R. Petasites hybridus extracts in vitro inhibit COX-2 and PGE2 release by direct interaction with the enzyme and by preventing ˜42144M A kinase activation in rat primary microglial cells. Planta Med 2005; 71:12-19.
[20] Murias M, Handler N, Erker T, Pleban K, Ecker G, Saiko P, Szekeres T, Jager W. Resveratrol analogues as selective cyclooxygenase-2 inhibitors: synthesis and structure-activity relationship. Bioorg Med Chem 2004; 12:5571-5578.
[21] [PI] Krotz F, Schiele T M, Klauss V, Sohn H Y. Selective COX-2 Inhibitors and Risk of Myocardial Infarction. J Vasc Res 2005; 42:312-324.
[22] Saliou C, Rimbach G, Moini H, McLaughlin L, Hosseini S, Lee J, Watson R R, Packer L. Solar ultraviolet-induced erythema in human skin and nuclear factor-kappa-B-dependent gene expression in keratinocytes are modulated by a French maritime pine bark extract. Free Radic Biol Med 2001; 30:154-160.

We claim:

1. A method for inhibiting COX-1 and COX-2 activity in a mammal in need thereof comprising administering to said mammal a composition comprising proanthocyanidins.

2. The method of claim 1 wherein said method reduces COX-1 activity by at least 10% and reduces COX-2 activities by at least 10%.

3. The method of claim 1 wherein said method increases the COX-1 inhibitory activity in the blood of said mammal by at least 10% and increases the COX-2 inhibitory activity in the blood of said mammal by at least 10%.

4. The method of claim 1 wherein said composition comprises an analgesic dose of proanthocyanidins.

5. The method of claim 1 wherein the composition is a composition for oral administration.

6. The method of claim 5 wherein said composition is in an oral form selected from the group consisting of pills, drinks, powders, food additives, powders, capsules, time-release-capsules.

7. The method of claim 1 wherein said administration comprises administering about 10 mg to about 10 grams per mammal per day of proanthocyanidins.

8. The method of claim 1 wherein said administration comprises administering about 20 mg to about 5 grams per mammal per day of proanthocyanidins.

9. The method of claim 1 wherein said administration comprises administering about 50 mg to about 1 gram per mammal per day of proanthocyanidins.

10. The method of claim 1 wherein said administrating comprises administering said proanthocyanidins daily for a period of at least 7 days.

11. The method of claim 1 wherein said administrating comprises administering said proanthocyanidins daily for a period of at least 14 days.

12. The method of claim 1 wherein said administrating comprises administering said proanthocyanidins daily for a period of at least 21 days.

13. The method of claim 1 wherein the proanthocyanidins are provided as an extract from a plant material.

14. The method of claim 13 where said plant extract is from a proanthocyanidins-rich plant and comprises between 10% to 100% proanthocyanidins.

15. The method of claim 13 where said plant extract is from a proanthocyanidins-rich plant and comprises between 25% to 100% proanthocyanidins.

16. The method of claim 13 where said plant extract is from a proanthocyanidins-rich plant and comprises between 60% to 100% proanthocyanidins.

17. The method of claim 13 said plant material is a bark of a pine tree.

18. The method of claim 17 wherein said pine bark is from Pinus pinaster.
